(19) United States
(12) Patent Application Publication
(10) Pub. No.: US 2024/0197798 A1
(43) Pub. Date: Jun. 20, 2024

(54) METHODS FOR INCREASING MENTAL WELL-BEING AND/OR HAPPINESS

(71) Applicant: Fonterra Co-Operative Group Limited, Auckland (NZ)

(72) Inventors: Rolando Saltini, Palmerston North (NZ); James Dekker, Waikanae (NZ); Maher Fuad, Palmerston North (NZ)

(21) Appl. No.: 18/543,604

(22) Filed: Dec. 18, 2023

(30) Foreign Application Priority Data
Dec. 16, 2022 (AU) 20232022903873
Jul. 21, 2023 (PH) 1-2023-050376
Jul. 21, 2023 (SG) 10202302077R
Jul. 21, 2023 (WO) PCT/IB2023/057433

(51) Int. Cl. A61K 35/747; A61K 35/00; A61K 45/06; A61P 25/24

(52) U.S. Cl. CPC A61K 35/747; A61K 45/06; A61P 25/24; A61K 2035/115

(57) ABSTRACT

The present invention provides a method of increasing mental well-being and/or happiness in a subject comprising administering an effective amount of a bacteria or a component thereof to a subject. The present invention also provides use of a bacteria or a component thereof in the manufacture of a medicament for increasing mental well-being and/or happiness in a subject and a composition comprising a bacteria or a component thereof for use in increasing mental well-being and/or happiness in a subject.

### FIELD OF THE INVENTION

The present invention provides a method of increasing mental well-being and/or happiness in a subject comprising administering an effective amount of a bacteria or a component thereof to a subject. The present invention also provides use of a bacteria or a component thereof in the manufacture of a medicament for increasing mental well-being and/or happiness in a subject and a composition comprising a bacteria or a component thereof for use in increasing mental well-being and/or happiness in a subject.

### BACKGROUND OF THE INVENTION

Over the years, the prevalence of mental health issues and Disability-adjusted life years (DALYs) attributed to mental health have been increasing (GBD Mental Disorders Collaborators, 2022). The COVID-19 pandemic is likely to have exacerbated the issue and more people started to suffer with their mental health (Chatterjee et al., 2020) and reduced happiness (Rossouw et al., 2021).

It is becoming increasingly clear that food habits can impact on brain health and mental well-being. However, the exact nature of these relationships remains to be elucidated. For instance, probiotics are beneficial microbes that offer health benefits when consumed in adequate quantities (FAO/WHO, 2001). Several in vitro and in vivo studies have been conducted to determine the effects of cognitive function of model systems and in humans. Some of these studies suggest that the consumption of probiotic formulations can improve cognitive function, stress management, and decision-making (Sivamaruthi et al., 2019). There has been little research on the effects of probiotics on happiness.

Considering the increase in the prevalence of mental health issues and reduced mental well-being in the human population (as well as their associated companion animals) there is a need for agents that promote mental well-being in a subject. Further, considering the reduction in happiness in the population there is an need for agents that promote happiness.

### SUMMARY OF THE INVENTION

The present inventors have developed methods, uses and compositions comprising Lacticaseibacillus rhamnosus strain HN001 or a derivative or component thereof for increasing mental well-being in a subject.

The present inventors have developed methods, uses and compositions comprising Lacticaseibacillus rhamnosus strain HN001 or a derivative or component thereof for increasing happiness in a subject.

In an aspect, the present invention provides a method of increasing mental well-being in a subject, the method comprising administering an effective amount of Lacticaseibacillus rhamnosus HN001 or a derivative or a component thereof to a subject.

In an aspect, the present invention provides a method of increasing happiness in a subject, the method comprising administering an effective amount of Lacticaseibacillus rhamnosus HN001 or a derivative or a component thereof to a subject.

In an aspect, the present invention provides a method of treating unhappiness in a subject, the method comprising administering an effective amount of Lacticaseibacillus rhamnosus HN001 or a derivative or a component thereof to a subject.

In an aspect, the present invention provides use of Lacticaseibacillus rhamnosus HN001 or a derivative or a component thereof in the manufacture of a medicament for increasing mental well-being in a subject.

In an aspect, the present invention provides use of Lacticaseibacillus rhamnosus HN001 or a derivative or a component thereof in the manufacture of a medicament for increasing happiness in a subject.

In an aspect, the present invention provides use of Lacticaseibacillus rhamnosus HN001 or a derivative or a component thereof in the manufacture of a medicament for treating unhappiness in a subject.

In an aspect, the present invention provides a composition comprising Lacticaseibacillus rhamnosus HN001 or a derivative or a component thereof for use in increasing mental well-being in subject.

In an aspect, the present invention provides a composition comprising Lacticaseibacillus rhamnosus HN001 or a derivative or a component thereof for use in increasing happiness in subject.

In an aspect, the present invention provides a composition comprising Lacticaseibacillus rhamnosus HN001 or a derivative or a component thereof for use in treating unhappiness in subject.

In an aspect, the present invention provides a method of increasing mental well-being in a subject, the method comprising administering an effective amount of Lacticaseibacillus rhamnosus comprising the AscI restriction enzyme digest profile of HN001 as shown in FIG. 5 or a component thereof to a subject.

In an aspect, the present invention provides a method of increasing happiness in a subject, the method comprising administering an effective amount of Lacticaseibacillus rhamnosus comprising the AscI restriction enzyme digest profile of HN001 as shown in FIG. 5 or a component thereof to a subject.

In an aspect, the present invention provides use of a Lacticaseibacillus rhamnosus comprising the AscI restriction enzyme digest profile of HN001 as shown in FIG. 5 or a component thereof in the manufacture of a medicament for increasing mental well-being in a subject.

In an aspect, the present invention provides use of a Lacticaseibacillus rhamnosus comprising the AscI restriction enzyme digest profile of HN001 as shown in FIG. 5 or a component thereof in the manufacture of a medicament for increasing happiness in a subject.

In an aspect, the present invention provides a composition comprising a Lacticaseibacillus rhamnosus comprising the AscI restriction enzyme digest profile of HN001 as shown in FIG. 5 or a component thereof for use in increasing mental well-being in subject.

In an aspect, the present invention provides a composition comprising a Lacticaseibacillus rhamnosus comprising the AscI restriction enzyme digest profile of HN001 as shown in FIG. 5 or a component thereof for use in increasing happiness in subject.

Any embodiment herein shall be taken to apply mutatis mutandis to any other embodiment unless specifically stated otherwise. For instance, as the skilled person would understand examples of prebiotics outlined for the methods of the invention equally apply to uses and compositions of the invention.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIGS. 1 & 2. Initial quality testing and probiotic viability. The Panels show viability pre-acid treatment, 15 minutes after acid treatment and 30 minutes after acid treatment.

FIG. 3. Flowchart of the study.

FIG. 4. Estimated marginal mean using Repeated measures ANOVA.

FIG. 5. Comparison of L. rhamnosus strains by PFGE of restriction-endonuclease-AscI digested genomic DNA: HN001 (lane 1); strain A (lane 2); Lactococcus lactis subsp. lactis strain ILI403 DNA digested with SmaI (lane 3); ATCC 53103 (lane 4); Lcr35 (lane 5); LGG (lane 6); ATCC 7469 (lane 7); ATCC 7469 (lane 8). Lane numbering is from left (1) to right (8).

FIG. 6. Shows the Oxford Happiness Score for female subjections taking the probiotic over a four week period. Repeated measures ANOVA (Treatment X Day). N=53 control; N=45 probiotic. Probiotic group had significantly higher happiness score; P=0.042.

FIG. 7. Shows the Oxford Happiness Score for male subjections taking the probiotic over a four week period. Repeated measures ANOVA (Treatment X Day). N=33 control; N=39 probiotic. No significant difference in happiness score; P=0.88.

FIG. 8. Shows the Oxford Happiness Score for male and female subjects taking the probiotic over a four week period. Repeated measures ANOVA (Treatment X Day). N=86 control; N=84 probiotic. Probiotic group tended towards higher happiness score; P=0.059. No significant effect of study centre (P=0.939).

### DETAILED DESCRIPTION

### General Techniques and Definitions

Unless specifically defined otherwise, all technical and scientific terms used herein shall be taken to have the same meaning as commonly understood by one of ordinary skill in the art (e.g., prebiotic).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “about”, unless stated to the contrary, refers to +/−10%, more preferably +/−5%, even more preferably +/−1%, of the designated value.

As used herein, the term “subject” is any animal. In one example, the animal is a vertebrate. For example, the animal is a mammal, avian, chordate, amphibian or reptile. In an embodiment, the subject is a mammal. In a preferred embodiment, the mammal is a human, a human companion animal or a human associated livestock. In an embodiment, the subject is an model animal, such as rat, mouse or guinea pig. In an embodiment, the mammal may be a companion animal, such as a cat, dog or horse. In an embodiment, the mammal may be livestock such as pigs, cattle, horses, goats, sheep, and deer.

In an embodiment, the subject is a human. In an embodiment, the subject is select from an/a: infant, child, adolescent, adult and elderly adult. As used herein an “elderly adult” is an adult of 65 years or older. In an embodiment, the subject is an infant. In an embodiment, the subject is a child. In an embodiment, the subject is an adolescent. In an embodiment, the subject is an adult. In an embodiment, the subject is an elderly adult. In an embodiment, the subject is characterised as not happy or somewhat unhappy using the Oxford Happiness Inventory (OHQ). In an embodiment, the subject is characterised as neutral, moderately happy or rather happy using the OHQ. In an embodiment, the subject has not been diagnosed as having any one or more of the following: anxiety, depression, stress, postnatal depression and postnatal anxiety. In an embodiment, the subject is not undergoing treatment, or has not been recommended treatment, for one or more of the following: anxiety, depression, stress, enhancing calm, occasional anxiety, immune suppression or dysfunction, vaginal health, prenatal depression, postnatal depression and postnatal anxiety. In an embodiment, the subject is not undergoing treatment, or has not been recommended treatment for anxiety. In an embodiment, the subject is not undergoing treatment, or has not been recommended treatment for depression. In an embodiment, the subject is not undergoing treatment, or has not been recommended treatment for stress. In an embodiment, the subject is not undergoing treatment, or has not been recommended treatment for enhancing calm. In an embodiment, the subject is not undergoing treatment, or has not been recommended treatment for occasional anxiety. In an embodiment, the subject is not undergoing treatment, or has not been recommended treatment for immune suppression or dysfunction. In an embodiment, the subject is not undergoing treatment, or has not been recommended treatment for vaginal health. In an embodiment, the subject is not undergoing treatment, or has not been recommended treatment for prenatal depression. In an embodiment, the subject is not undergoing treatment, or has not been recommended treatment for postnatal depression. In an embodiment, the subject is not undergoing treatment, or has not been recommended treatment for postnatal anxiety.

In an embodiment, the subject is female. In an embodiment, the subject is pregnant or postpartum. In an embodiment, the subject is prenatal. In an embodiment, the female is not pregnant. In an embodiment, the female has not been pregnant. In an embodiment, the female is not postpartum. In an embodiment, the subject is not prenatal.

In an embodiment, the subject is a male.

As used herein, “microorganism” or “microorganisms” refers to microscopic organisms including bacterial, viral, fungal or eukaryotic organisms.

As used herein, “probiotic” refers to live microorganism which when administered in an adequate amount confers a health benefit to a subject as described herein.

As used herein, “psychobiotic” refers to a class of probiotics which produce mental health benefits in a subject (Sharma et al., 2021).

As used herein, “prebiotic” refers to a substrate that is selectively utilized by host microorganisms conferring a health benefit.

As used herein, a “postbiotic” refers to a preparation of inanimate microorganisms and/or a component of the microorganism that confers a health benefit on the host directly or indirectly (e.g. as described in Vinderola et al., 2022). In an embodiment, the inanimate microorganism confers the health benefit to the host directly or indirectly. In an embodiment, the inanimate microorganism confers the health benefit to the host directly. In an embodiment, the inanimate microorganism confers the health benefit to the host indirectly. In an embodiment, one or more components of the microorganism confers the health benefit to the host. In an embodiment, the postbiotic comprises a microorganism that has been killed (e.g. by heat or chemical treatment).

As used herein “a component”, “component of the microorganism” and “postbiotic component” refers to a part (fragment) of the cell or a product secreted from a cell. Examples of postbiotic components are described for example in Zółkiewicz et al. (2020) and Vinderola et al. (2022). In an embodiment, the component is selected from one or more of: a cell wall fragment, cell supernatant, cell lysate, intracellular metabolite, secreted metabolite, enzyme, protein, short chain fatty acid, vitamin, phenol, amino acid, bacteriocin, extracellular vesicle, and exopolysaccharide. In an embodiment, the component is a cell wall component. In an embodiment, the component is a cell free supernatant. In an embodiment, the component is a cell lysate. In an embodiment, the component is an intracellular metabolite. In an embodiment, the component is an extracellular metabolite. In an embodiment, the component is an enzyme. In an embodiment, the component is a protein. In an embodiment, the component is a short chain fatty acid. In an embodiment, the component is a vitamin. In an embodiment, the component is a phenol. In an embodiment, the component is an amino acid. In an embodiment, the component is a bacteriocin. In an embodiment, the component is an extracellular vesicle. In an embodiment, the component is an exopolysaccharide. In embodiment, the secretion is conditioned medium obtained from culturing the bacteria.

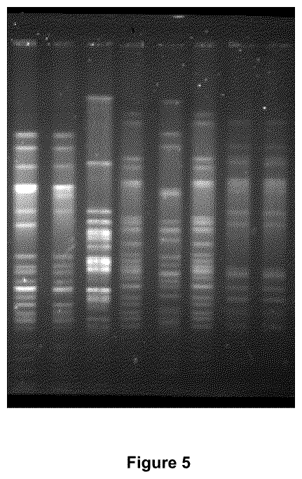

As used herein, the term “derivative” or “derivatives” of Lacticaseibacillus rhamnosus HN001 refers to mutants and closely related strain of HN001. In an embodiment, the derivative has the same restriction enzyme profile as HN001 (see for example the AscI restriction enzyme profile shown in FIG. 5). In an embodiment, the derivative is plasmid free (e.g. as described in Zhou et al., 2005).

As used herein, the term “increase” or “increases” or “increased” or “increasing” refers to having a higher or greater level of a given parameter after application of the methods, uses or compositions as described herein compared to the level of a given parameter at baseline.

As used herein, the terms “treat”, “treating” or “treatment” refers to at least partially obtaining a desired therapeutic outcome. In an embodiment, treating comprises preventing or delaying the appearance of one or more symptoms of unhappiness. In an embodiment, treating comprises arresting or reducing the development of one or more symptoms of unhappiness. In an embodiment, treating comprises increasing happiness in a subject characterised as “not happy” or “unhappy” for example using the Oxford Happiness Inventory.

### HN001

As described in PCT publication No. WO 99/10476, Lacticaseibacillus rhamnosus HN001 is available under Australian Government Analytical Laboratories (AGAL), deposit number NM97/09514, dated 18 Aug. 1997. This Budapest Treaty-recognised depository is now no longer referred to as AGAL, but rather is referred to as the National Measurement Institute of Australia (NMIA) located at 1/153 Bertie Street, Port Melbourne, Victoria, Australia 3207.

HN001 has been used as described in, for example, WO 99/10476 and WO 2018/220429, for conditions other than increasing happiness. HN001 is described for example in Slykerman et al. (2017) and Tay et al. (2020). HN001 is commercially available from NZMP, SureStart™, the early life nutrition brand and Nutiani™, the health & wellness solutions brand of dairy co-operative, Fonterra. HN001 has also been marketed by the names LactoB HN001™, Nutiani HN001™ and DR20™. In an embodiment, HN001 is administered in a reproductively viable form. In an embodiment, a proportion of the HN001 is killed, lysed, fractionated or attenuated. In an embodiment, HN001 is killed, lysed, fractionated or attenuated.

### Well-Being

The present invention provides methods, uses and composition comprising Lacticaseibacillus rhamnosus HN001 or a derivative or a component thereof for increasing well-being in a subject. As used herein, and as defined by the American Psychological Association (APA), “well-being” is a state of happiness and contentment, with low levels of distress, overall good physical and mental health and outlook, or good quality of life. In some embodiment, the well-being is mental well-being. As used herein, “mental well-being” is where individuals can cope with the normal stresses of life and contribute to her or his community.

In an embodiment, mental well-being in increased in a subject by about 5% to about 10%, or about 5% to about 15%, or about 5% to about 20%, or about 5% to about 25%, or about 5% to about 30%, or about 5% to about 35%, or about 5% to about 45%, or about 5% to about 55%, or about 5% to about 65%, or about 5% to about 75%, or about 5% to about 85%, or about 5% to about 95%, or about 100% compared to baseline. In an embodiment, mental well-being in increased in a subject by about 5% to about 10% compared to baseline. In an embodiment, mental well-being in increased in a subject by about 5% to about 15% compared to baseline. In an embodiment, mental well-being in increased in a subject by about 5% to about 20% compared to baseline. In an embodiment, mental well-being in increased in a subject by about 5% to about 30% compared to baseline. In an embodiment, mental well-being in increased in a subject by about 5% to about 35% compared to baseline. In an embodiment, mental well-being in increased in a subject by about 5% to about 45% compared to baseline. In an embodiment, mental well-being in increased in a subject by about 5% to about 55% compared to baseline. In an embodiment, mental well-being in increased in a subject by about 5% to about 65% compared to baseline. In an embodiment, mental well-being in increased in a subject by about 5% to about 75% compared to baseline. In an embodiment, mental well-being in increased in a subject by about 5% to about 85% compared to baseline. In an embodiment, mental well-being in increased in a subject by about 5% to about 95% compared to baseline. In an embodiment, mental well-being in increased in a subject by about 100% compared to baseline.

In an embodiment, mental well-being in increased in a subject by at least 5%, or at least 10%, or at least 15%, or at least 20%, or at least 25%, or at least 30%, or at least 35%, or at least 40%, or at least 45%, or at least 50%, or at least 60%, or at least 70%, or at least 80%, or at least 90%, or at least 100% compared to baseline.

In an embodiment, mental well-being is increased in a subject by improving cognitive brain function. In an embodiment, mental well-being is increased in a subject by improving happiness.

In an embodiment, mental well-being is assessed using the Warwick-Edinburgh Mental Wellbeing Scale (WEMWBS) (Tennant et al., 2007). In an embodiment, mental well-being is assessed using the Short Warwick-Edinburgh Mental Wellbeing Scale (SWEMWBS) (Stewart-Brown et al., 2009).

### Happiness

The present invention provides methods, uses and composition comprising Lacticaseibacillus rhamnosus HN001 or a derivative or a component thereof for increasing happiness in a subject. As used herein, and as defined by the American Psychological Association (APA), “happiness” or “happy” is an emotion of joy, gladness, satisfaction, and well-being.

A person skilled in the art will appreciate that happiness can be assessed using any happiness tool known to a person skilled in the art, including, for example the Oxford Happiness Inventory (OHQ) (Argyle et al., 1989); Subjective Happiness scale (SHS) (Lyubomirsky and Lepper, 1999); Satisfaction with Life Scale (SLS) (Diener et al (1985); Panas Scale (PS) (Watson et al., 1988); Authentic Happiness Inventory (AHI) (Seligman, 2002); Psychological Wellbeing Scale (PWS) (Ryff et al., 2007; adapted from Ryff, 1989) and Fordyce Emotions Questionnaire (FEQ) (Fordyce, 1988). In an embodiment, happiness is assessed using the OHQ. The OHQ is described in further detail in Example 1 of the specification. The OHQ has a total of 29 items. Each item is scored on a Likert scale from 1 to 6 (1=strongly disagree; 2=moderately disagree; 3=slightly disagree; 4=slightly agree; 5=moderately agree; 6=strongly agree). Some items are phrased positively and others negatively. Such that negative items (1, 3, 12, 13, 16, 18, 21 and 29) should be scored in reverse (Hills and Argyle, 2002). The total score is then is divided by 29. Interpretation of the final score is as follows: A final score between 1 to 2 is considered “not happy”; between 2 and 3 is “somewhat unhappy”; between 3 and 4 is considered “neutral”; 4 is “somewhat or moderately happy”; between 4 and 5 is “rather happy”; between 5 and 6 is “very happy”; 6 is “too happy” (Hills and Argyle, 2002).

In an embodiment, happiness is assessed using the SHS. In an embodiment, happiness is assessed using the SLS. In an embodiment, happiness is assessed using the PS. In an embodiment, happiness is assessed using the AHI. In an embodiment, happiness is assessed using the PWS. In an embodiment, happiness is assessed using the FEQ.

In an embodiment, happiness in increased in a subject by about 5% to about 10%, or about 5% to about 15%, or about 5% to about 20%, or about 5% to about 25%, or about 5% to about 30%, or about 5% to about 35%, or about 5% to about 45%, or about 5% to about 55%, or about 5% to about 65%, or about 5% to about 75%, or about 5% to about 85%, or about 5% to about 95%, or about 100% compared to baseline. In an embodiment, happiness in increased in a subject by about 5% to about 10% compared to baseline. In an embodiment, happiness in increased in a subject by about 5% to about 15% compared to baseline. In an embodiment, happiness in increased in a subject by about 5% to about 20% compared to baseline. In an embodiment, happiness in increased in a subject by about 5% to about 30% compared to baseline. In an embodiment, happiness in increased in a subject by about 5% to about 35% compared to baseline. In an embodiment, happiness in increased in a subject by about 5% to about 45% compared to baseline. In an embodiment, happiness in increased in a subject by about 5% to about 55% compared to baseline. In an embodiment, happiness in increased in a subject by about 5% to about 65% compared to baseline. In an embodiment, happiness in increased in a subject by about 5% to about 75% compared to baseline. In an embodiment, happiness in increased in a subject by about 5% to about 85% compared to baseline. In an embodiment, happiness in increased in a subject by about 5% to about 95% compared to baseline. In an embodiment, happiness in increased in a subject by about 100% compared to baseline.

In an embodiment, happiness in increased in a subject by at least 5%, or at least 10%, or at least 15%, or at least 20%, or at least 25%, or at least 30%, or at least 35%, or at least 40%, or at least 45%, or at least 50%, or at least 60%, or at least 70%, or at least 80%, or at least 90%, or at least 100% compared to baseline.

In an embodiment, increasing happiness comprises increasing a subjects score on the OHQ. For example, a subject with a OHQ baseline score of 1 is increased to score greater than 1, or a subject with a baseline score of 2 is increase to a score greater than 2, or a subject with a baseline score of 3 is increased to a score greater than 3, or a subject with a baseline scope of 4 is increased to a score greater than 4 after application of the methods, uses or compositions as described herein.

In an embodiment, increasing happiness comprises increasing happiness in a neutral or happy subject. In an embodiment, at baseline the subject is characterised as neutral, moderately happy or rather happy using the OHQ. In an embodiment, increasing happiness comprises increasing a subject with a baseline OHQ score of 3 to a score greater than 3. In an embodiment, increasing happiness comprises increasing a subject with a baseline OHQ score of 4 to a score greater than 4. In an embodiment, increasing happiness comprises increasing a subject with a baseline OHQ score of 5 to a score greater than 5.

In an embodiment, increasing happiness comprises treating unhappiness in a subject. In an embodiment, at baseline the subject is characterised as not happy or somewhat unhappy using the OHQ. In an embodiment, treating unhappiness comprises increasing a subject with a baseline OHQ score of 1 to a score greater than 1. In an embodiment, treating unhappiness comprises increasing a subject with a baseline OHQ score of 2 to a score greater than 2.

In an embodiment, increasing happiness in a subject comprises changes in levels of salivary cortisol. In an embodiment, the change in level of salivary cortisol is a decrease in the level of salivary cortisol. In an embodiment, increasing happiness in a subject does not comprise changes in levels of salivary cortisol.

A person skilled in the art will appreciate that happiness is a parameter that can be measured separately from anxiety, depression and stress. In an embodiment, increasing happiness does not comprise reducing anxiety. In an embodiment, increasing happiness does not comprise reducing depression. In an embodiment, increasing happiness does not comprise reducing stress. In an embodiment, increasing happiness does not comprise reducing postnatal depression. In an embodiment, increasing happiness does not comprise reducing postnatal anxiety.

In an embodiment, the method increasing happiness additional comprises dietary modulation. In an embodiment, the method of increasing happiness does not comprise dietary modulation.

### Stress

As used herein, and as defined by the APA, “stress” is the physiological or psychological response to internal or external stressors. Stress involves changes affecting nearly every system of the body, influencing how people feel and behave. For example, it may be manifested by palpitations, sweating, dry mouth, shortness of breath, fidgeting, accelerated speech, augmentation of negative emotions (if already being experienced), and longer duration of stress fatigue. Severe stress is manifested by the general adaptation syndrome. By causing these mind-body changes, stress contributes directly to psychological and physiological disorder and disease and affects mental and physical health, reducing quality of life.

### Anxiety

As used herein, and as defined by the APA, “anxiety” is as an emotion characterised by apprehension and somatic symptoms of tension in which an individual anticipates impending danger, catastrophe, or misfortune. The body often mobilises itself to meet the perceived threat: Muscles become tense, breathing is faster, and the heart beats more rapidly.

Both stress and anxiety are emotional responses, however stress is typically caused by an external trigger. The trigger can be short-term, such as a work deadline or a fight with a loved one or long-term, such as being unable to work, discrimination, or chronic illness. Anxiety, on the other hand, is defined by persistent, excessive worries that do not go away even in the absence of a stressor.

### Depression

As used herein, and as defined by the APA, “major depressive disorder” or “depression” is a serious mood disorder typified by persistent feelings of sadness, emptiness, pessimism and/or low mood that negatively impacts daily life activities, including eating, and working. Symptoms include feelings of hopelessness, irritability, guilt, worthlessness, or helplessness. It can also include loss of interest or pleasure in leisure activities, decreased energy, fatigue, or restlessness, and can lead to difficulties with concentration and focus, memory, and decision making. Depression can also disrupt normal appetite and/or weight, and can result in suicide, or attempted suicide. In addition, depression can involve digestive discomfort and other digestive problems, and other aches or pains without a clear physical cause. While postnatal depression (PND; also known as perinatal depression or postpartum depression) share a number of features with major depressive episodes, PND is typified by a series of features that makes it a separate entity from major depressive disorder, over and above the obvious association between PND and pregnancy/childbirth. For example, PND is associated with hormonal changes specific to the transition from pregnancy to the postpartum period. Animal model experiments have shown that rats given hormones to mimic this transition develop behavioural changes typical of PND. In addition, PND is associated with specific psychosocial stressors including infant temperament, and the stress associated with caring for a new-born infant. In contrast to typical patients with major depressive disorder, PND sufferers exhibit decreased activation of many neural regions, such as a blunted amygdala response to negative stimuli not related to the infant, or reduced emotional responses and regulation in response to their own infant's cries.

### Calmness

As used herein “calmness” or “calm” is the mental state of peace of mind being free from agitation, excitement, or disturbance. It also refers to being in a state of serenity, tranquility, or peace. Calmness can most easily occur for the average person during a state of relaxation, but it can also be found during much more alert and aware states.

### Other States/Conditions

In addition, or alternatively the methods, uses and compositions as described herein can be used to effect one or more of the following in a subject: increase cognitive function; improve mood in a subject; and reduce cognitive fatigue.

In addition, or alternatively the methods, uses and compositions as described herein increases cognitive function in a subject. As used herein, and as defined by the APA, “cognitive function” is the performance of the mental processes of perception, learning, memory, understanding, awareness, reasoning, judgment, intuition, and language. In addition, or alternatively the methods, uses and compositions as described herein improves mood in a subject. In addition, or alternatively the methods, uses and compositions as described herein reduce cognitive fatigue.

### Compositions and Products

In an aspect, the present invention provides a composition comprising Lacticaseibacillus rhamnosus HN001 or a derivative or a component thereof for use in increasing mental well-being in subject. In an aspect, the present invention provides a composition comprising Lacticaseibacillus rhamnosus HN001 or a derivative or a component thereof for use in increasing happiness in subject. In an embodiment, the composition is a food product or nutritional supplement.

In an embodiment, the composition is a pharmaceutical composition.

In an embodiment, the composition is a probiotic composition. In an embodiment, the probiotic composition is a psychobiotic composition. In an embodiment, the composition is a postbiotic composition. In an embodiment, the composition further comprises a prebiotic. In an embodiment, the composition is a combined prebiotic and probiotic composition. In an embodiment, the composition is a combined prebiotic and postbiotic composition. In an embodiment, the composition is a synbiotic composition.

In an embodiment, the composition comprises one or more other probiotic or postbiotic microorganism/s. In an embodiment, the composition as described herein is suitable for administration to a human.

In an embodiment, the composition is in a form selected from a: tablet, powder, capsule, liquid, syrup, gummy and a sachet.

In an embodiment, the composition comprises one or more of: a milk lipid, phospholipids; powdered or fresh skim milk, recombined or reconstituted whole or skim milk powder, skim milk concentrate, skim milk retentate, concentrated milk, ultrafiltered milk retentate, milk protein concentrate (MPC), milk protein isolate (MPI), calcium depleted milk protein concentrate (MPC), low fat milk, low fat milk protein concentrate (MPC), casein, caseinate, milk fat, cream, butter, ghee, anhydrous milk fat (AMF), buttermilk, butter serum, beta serum, hard milk fat fractions, soft milk fat fractions, sphingolipid fractions, milk fat globular membrane fractions, milk fat globular membrane lipid fractions, phospholipid fractions, complex lipid fractions, milk extracellular vesicle fractions, colostrum, a colostrum fraction, colostrum protein concentrate (CPC), colostrum whey, an immunoglobulin fraction from colostrum, whey (including sweet whey, lactic acid whey, mineral acid whey, or reconstituted whey powder), whey protein isolate (WPI), whey protein concentrate (WPC including whey protein phospholipid concentrate (WPPC)), glycomacropeptide, lactoferrin, iron-lactoferrin, a functional lactoferrin variant, a functional lactoferrin fragment, a vitamin D, calcium, a composition derived from any milk or colostrum processing stream, blueberry, bilberry, blackcurrant, extracellular vesicles, miRNA, polyunsaturated fatty acids, curcumin, green tea, fucoidan, soy isoflavone, conjugated linoleic acid, and composition derived from the retentate or permeate obtained by ultrafiltration or microfiltration of any milk or colostrum processing stream.

The composition may also include other nutrients, such as proteins, carbohydrates, vitamins, minerals, or amino acids. In an embodiment, the amino acid is arginine and/or glutamine.

In an embodiment, the composition comprises one or more of coconut powder, vitamin C and calcium. In an embodiment, the composition comprises coconut powder. In an embodiment, the composition comprises vitamin C. In an embodiment, the composition comprises calcium.

The food product may be any edible consumer product which is able to carry Lacticaseibacillus rhamnosus HN001 or a derivative or a component thereof. Examples of such consumer products include the following non-limiting examples: cultured milk, yoghurt, cheese, milk and yoghurt drinks, or milk powder, baby food, fruit juice, rice pudding, rusks, purees, cereals, porridge, confectionary products, reconstituted fruit products, snack bars, food bars, muesli bars, spreads, sauces, dips, sports supplements including dairy and non-dairy based sports supplements, food additives such as protein sprinkles, dietary supplement products including daily supplement tablets, weaning foods and yoghurts, and formulas such as maternal formula, in powder or liquid form, including hypoallergenic embodiments of such compositions.

In an embodiment, the food product is a dairy product. In an embodiment, the dairy product is selected from: milk, yoghurt, drinking yoghurt, butter, cream, ice-cream and cheese. In an embodiment, the food product is a dairy mimic. In an embodiment, the food product or nutritional supplement is selected from: an infant formula, a follow-on formula, a growing-up formula, a maternal formula, a maternal supplement, an adult nutritional formulation, and an elderly nutritional formulation.

Flavourants, colourants, and other additives, carriers or excipients, as would be well known to those skilled in the art, may also be included in the compositions of the invention. It will be appreciated that a broad range of additives or carriers may be included in such compositions or food products, for example to improve or preserve bacterial viability or to increase therapeutic efficacy of Lacticaseibacillus rhamnosus HN001 or derivatives thereof. For example, additives such as surfactants, wetters, humectants, stickers, dispersal agents, stabilisers, penetrants, and so-called stressing additives to improve bacterial cell vigour, growth, replication and survivability (such as potassium chloride, glycerol, sodium chloride and glucose), as well as cryoprotectants such as maltodextrin, may be included. Additives may also include compositions which assist in maintaining microorganism viability in long term storage, for example unrefined corn oil, or “invert” emulsions containing a mixture of oils and waxes on the outside and water, sodium alginate and bacteria on the inside.

The composition of the invention may comprise live Lacticaseibacillus rhamnosus HN001 or a derivative thereof, preferably in a reproductively viable form and amount. Methods to produce such compositions are well-known in the art. The composition may comprise a carbohydrate source, such as a disaccharide including, for example, sucrose, fructose, glucose, or dextrose. Preferably the carbohydrate source is one able to be aerobically or anaerobically utilised by Lacticaseibacillus rhamnosus Lacticaseibacillus rhamnosus HN001 or a derivative thereof. The composition is preferably capable of supporting reproductive viability of Lacticaseibacillus rhamnosus HN001 or a derivative thereof for a period greater than about two weeks, preferably greater than about one month, about two months, about three months, about four months, about five months, more preferably greater than about six months, most preferably at least about 2 years to about 3 years or more.

Methods to produce the compositions of the invention are well-known in the art, and may use standard microbiological and pharmaceutical practices.

In one embodiment, the composition comprises about 0.1, 0.2, 0.5, 1, 5, 10, 15, 20, 25, 30, 35, 40, 45, 50, 55, 60, 65, 70, 75, 80, 85, 90, 95, 99, 99.5, 99.8 or 99.9% by weight of Lacticaseibacillus rhamnosus HN001 or a derivative or component thereof.

It will be apparent that the concentration of Lacticaseibacillus rhamnosus HN001 or a derivative thereof in a composition formulated for administration may be less than that in a composition formulated for, for example, distribution or storage, and that the concentration of a composition formulated for storage and subsequent formulation into a composition suitable for administration must be adequate to allow said composition for administration to also be sufficiently concentrated so as to be able to be administered at a therapeutically efficacious dose.

### Other Probiotic or Postbiotic Microorganism/s

A person skilled in the art will appreciate that the methods, compositions and products as described herein may comprise one or more other probiotic or postbiotic microorganism/s. In an embodiment, the one or more other probiotic or postbiotic microorganism/s has beneficial effects on one or more of: the respiratory tract, the digestive tract and the skin. In an embodiment, the one or more other probiotic or postbiotic microorganism/s is a yeast or bacteria.

In an embodiment, the one or more other probiotic or postbiotic microorganism/s is/are selected from a: Acetilactobacillus, Agrilactobacillus, Akkermansia, Amylolactobacillus, Anaerobutyricum, Apilactobacillus, Bacillus, Bacteroides, Bombilactobacillus, Christensenella, Clostridium, Companilactobacillus, Dellaglioa, Enterococcus, Eubacterium, Faecalibacterium, Fructilactobacillus, Furfurilactobacillus, Holzapfelia, Lacticaseibacillus, Lactiplantibacillus, Lactococcus, Lapidilactobacillus, Latilactobacillus, Lentilactobacillus, Leuconostoc, Levilactobacillus, Ligilactobacillus, Limosilactobacillus, Liquorilactobacillus, Loigolactobacilus, Parabacteroides, Paucilactobacillus, Pediococcus, Prevotella, Schleiferilactobacillus, Secundilactobacillus, and Streptococcus.

In an embodiment, the one or more other probiotic or postbiotic microorganism/s is/are selected from a: Akkermansia muciniphila, Anaerobutyricum hallii, Bacillus coagulans, Bacillus subtilis, Bacteroides fragilis, Bacteroides thetaiotaomicron, Bacteroides uniformis, Bifidobacterium adolescentis, Bifidobacterium animalis, Bifidobacterium bifidum, Bifidobacterium breve, Bifidobacterium infantis, Bifidobacterium longum, Bifidobacterium longum, Christensenella minuta, Clostridium butyricum, Enterococcus durans, Enterococcus faecalis, Enterococcus faecium, Faecalibacterium prausnitzii, Lacticaseibacillus casei, Lacticaseibacillus paracasei, Lacticaseibacillus rhamnosus, Lactiplantibacillus pentosus, Lactiplantibacillus plantarum, Lactobacillus acidophilus, Lactobacillus bulgaricus, Lactobacillus crispatus, Lactobacillus gallinarum, Lactobacillus gasseri, Lactobacillus helveticus, Lactobacillus johnsonii, Lactococcus lactis, Latilactobacillus sakei, Lentilactobacillus kefiri, Leuconostoc mesenteroides, Levilactobacillus brevis, Ligilactobacillus salivarius, Limosilactobacillus fermentum, Limosilactobacillus reuteri, Parabacteroides goldsteinii, Pediococcus acidilactici, Pediococcus pentosaceus, Prevotella copri, Saccharomyces boulardii, and Streptococcus thermophilus.

In an embodiment, the one or more other lactic acid bacteria is/are selected from: L. reuteri RC-14, B. longum BB536, L. lactis MG1363, L. plantarum LP299V, L. plantarum LP-115, B. lactis HN019, L. reuteri JCM 1112, L. casei ATCC393, B. animalis B94, L. paracasei LPC-37, L. acidophilus NCFM, B. lactis BL-04, B. lactis Bi-07, L. crispatus LBV88, L. gasseri LG-36, B. longum CCFM1029, L. rhamnosus HN067, L. rhamnosus GG (LGG), L. acidophilus LA-14, B. breve M-16V, B. lactis BB12 and L. acidophilus HN017.

In an embodiment, the one or more other lactic acid bacteria is/are selected from: L. reuteri RC-14, B. longum BB536, L. lactis MG1363, L. plantarum LP299V, L. plantarum LP-115, B. lactis HN019, L. reuteri JCM 1112, L. casei ATCC393, B. animalis B94, L. paracasei LPC-37, L. acidophilus NCFM, B. lactis BL-04, B. lactis Bi-07, L. crispatus LBV88, L. gasseri LG-36, B. longum CCFM1029, L. rhamnosus HN067, L. rhamnosus GG (LGG), B. breve M-16V, B. lactis BB12 and L. acidophilus HN017.

In an embodiment, the one or more other lactic acid bacteria is reuteri RC-14. In an embodiment, the one or more other lactic acid bacteria is B. longum. In an embodiment, the one or more other lactic acid bacteria is BB536. In an embodiment, the one or more other lactic acid bacteria is L. lactis MG1363. In an embodiment, the one or more other lactic acid bacteria is L. plantarum LP299V. In an embodiment, the one or more other lactic acid bacteria is L. plantarum LP-115. In an embodiment, the one or more other lactic acid bacteria is B. lactis HN019. In an embodiment, the one or more other lactic acid bacteria is L. reuteri JCM 1112. In an embodiment, the one or more other lactic acid bacteria is L. casei ATCC393. In an embodiment, the one or more other lactic acid bacteria is B. animalis B94. In an embodiment, the one or more other lactic acid bacteria is L. paracasei LPC-37. In an embodiment, the one or more other lactic acid bacteria is L. acidophilus NCFM. In an embodiment, the one or more other lactic acid bacteria is B. lactis BL-04. In an embodiment, the one or more other lactic acid bacteria is B. lactis Bi-07. In an embodiment, the one or more other lactic acid bacteria is L. crispatus LBV88. In an embodiment, the one or more other lactic acid bacteria is L. gasseri LG-36. In an embodiment, the one or more other lactic acid bacteria is B. longum CCFM1029. In an embodiment, the one or more other lactic acid bacteria is L. rhamnosus HN067. In an embodiment, the one or more other lactic acid bacteria is L. rhamnosus GG (LGG). In an embodiment, the one or more other lactic acid bacteria is B. breve M-16V. In an embodiment, the one or more other lactic acid bacteria is B. lactis BB12. In an embodiment, the one or more other lactic acid bacteria is L. acidophilus HN017.

In an embodiment, the one or more other lactic acid bacteria is not L. acidophilus LA-14.

In an embodiment, the one or more other probiotics is a psychobiotic. Examples of psychobiotics are described for example in Sharma et al. (2021).

In an embodiment, the methods, composition and products as described herein may comprise one or more prebiotics. Examples of prebiotics are described for example in Davani-Davari et al. (2019). In an embodiment, the prebiotic is selected from one or more of: galacto-oligosaccharide, fructo-oligosaccharide, trans-galacto-oligosaccharide, inulin, a human milk oligosaccharide, gentio-oligosaccharide, xylo-oligosaccharide, isomalto-oligosaccharide, bran, resistant starch, lactulose, lactitol, flavanols and pectic oligosaccharide. In an embodiment, the prebiotic is a human milk oligosaccharide (e.g. as described in Corona et al., 2021). In an embodiment, the human milk oligosaccharide is selected from one or more of: 2′-Fucosyllactose (2′FL), (3-fucosyllactose) 3FL, (lacto-N-tetraose) LNT, (Lacto-N-neotetraose) LNnT, (6-′sialyllactose) 6′SL, (3′-sialyllactose) 3′SL, (6′-sialyllactosamine) 6′SLN, difucosyllactose (DFL).

### Administration

A variety of routes of administration are possible for methods, uses and composition as described herein, including but not limited to oral, dietary, rectal and topical administration. In an embodiment, administration is oral administration. In an embodiment, administration is rectal administration selected from one or more of: suppository, enema, via colonoscope or other medical equipment and faecal transplantation. In an embodiment, administration is topical administration.

In an embodiment, administration is daily. In an embodiment, administration is twice daily. In an embodiment, administration is three times per day. In an embodiment, administration is for about 17 days to about 80 days. In an embodiment, administration is for about 17 days to about 70 days. In an embodiment, administration is for about 20 days to about 65 days. In an embodiment, administration is for about 25 days to about 65 days. In an embodiment, administration is for about 30 days to about 60 days. In an embodiment, administration is for at least about 30 days. In an embodiment, administration is for at least 60 about days.

In an embodiment, administration is for about 2 weeks to about 10 weeks. In an embodiment, administration is for about 3 weeks to about 9 weeks. In an embodiment, administration is for about 4 weeks to about 8 weeks. In an embodiment, administration is for at least 4 weeks, or 5 weeks, or 6 weeks, or at least 7 weeks, or at least 8 weeks. In an embodiment, administration is for at least 4 weeks. In an embodiment, administration is for at least 5 weeks. In an embodiment, administration is for at least 6 weeks. In an embodiment, administration is for at least 7 weeks. In an embodiment, administration is for at least 8 weeks.

In an embodiment, administration is in the range of about 1×10⁵ to about 1×10¹² HN001 or a derivative thereof colony forming units (CFU) daily. In an embodiment, administration is in the range of about 1×10⁵ to about 1×10¹¹ HN001 or a derivative thereof CFU daily. In an embodiment, administration is in the range of about 1×10⁵ to about 1×10¹⁰ HN001 or a derivative thereof CFU daily. In an embodiment, administration is in the range of about 1×10⁶ to about 1×10¹⁰ HN001 or a derivative thereof CFU daily. In an embodiment, administration is in the range of about 1×10⁷ to about 1×10¹⁰ HN001 or a derivative thereof CFU daily. In an embodiment, administration is in the range of about 1×10⁸ to about 1×10¹⁰ HN001 or a derivative thereof CFU daily. In an embodiment, administration is in the range of about 2×10⁹ to about 1×10¹⁰ HN001 or a derivative thereof CFU daily. In an embodiment, administration is in the range of about 4×10⁹ to about 1×10¹⁰ HN001 or a derivative thereof CFU daily. In an embodiment, administration is in the range of about 5×10⁹ to about 1×10¹⁰ HN001 or a derivative thereof CFU daily. In an embodiment, administration is in the range of about 6×10⁹ to about 1×10¹⁰ HN001 or a derivative thereof CFU daily. In an embodiment, administration is in the range of about 6×10⁹ to about 9.5×10⁹ HN001 or a derivative thereof CFU daily. In an embodiment, administration comprises about 6×10⁹ CFU daily. In an embodiment, administration comprises about 9.5×10⁹ CFU daily. In an embodiment, administration comprises about 9.5×10¹⁰ CFU daily.

In an embodiment, administration is in the range of about 2×10⁹ to about 1×10¹⁰ HN001 or a derivative thereof CFU daily for about 17 to about 80 days. In an embodiment, administration is in the range of about 3×10⁹ to about 1×10¹⁰ HN001 or a derivative thereof CFU daily for about 17 to about 80 days. In an embodiment, administration is in the range of about 4×10⁹ to about 9.8×10⁹ HN001 or a derivative thereof CFU daily for about 17 to about 80 days. In an embodiment, administration is in the range of about 5×10⁹ to about 9.7×10⁹ HN001 or a derivative thereof CFU daily for about 17 to about 80 days. In an embodiment, administration is in the range of about 6×10⁹ to about 9.5×10⁹ HN001 or a derivative thereof CFU daily for about 17 to about 80 days. In an embodiment, administration comprises about 6×10⁹ HN001 or a derivative thereof CFU daily for about 17 to about 80 days. In an embodiment, administration comprises about 9×10⁹ HN001 or a derivative thereof CFU daily for about 17 to about 80 days.

As a person skilled in the art will appreciate a probiotic may be manufactured at a set CFU but the viability of the probiotic can decline over time. In some cases the viability of the probiotic can decline by up to 40% over a 12 month period. The above administration ranges define viable cells delivered to a subject.

In some embodiments, the composition is manufactured to comprise about 9×10⁹ HN001 or a derivative thereof CFU daily to ensure delivery of at least about 6×10⁹ HN001 or a derivative thereof CFU daily.

When used in combination with another therapeutic agent (e.g. one or more other probiotic, postbiotic, prebiotic or psychobiotic), the administration of a composition useful herein and the other therapeutic agent may be simultaneous or sequential. Simultaneous administration includes the administration of a single dosage form that comprises all components or the administration of separate dosage forms at substantially the same time. Sequential administration includes administration according to different schedules, preferably so that there is an overlap in the periods during which the composition useful herein and other therapeutic agent(s) are provided.

### EXAMPLES

### Example 1—Effect of HN001 on Happiness

Over the years, the prevalence of mental health issues and Disability-adjusted life years (DALYs) attributed to mental health have been increasing. The objectives of the current study were to investigate the survival of the HN001 in acid media and to analyse customer feedback following the ingestion of a specifically constructed probiotic intervention to evaluate the role of HN001 on mental wellness, specifically their happiness scores over a period of 60 days. Capsules containing probiotic strain L. rhamnosus HN001 at a dose of 6×10⁹ colony forming units (CFU) daily were administered. The primary outcome was a change in Oxford happiness questionnaire (OHQ) scores after 30 and 60 days respectively. Fifty-eight participants were consulted and selected for the study. Initial quality testing showed that the probiotics survived the acidic environment. Over a period of 60 days the Oxford happiness questionnaire scores improved (P value <001).

### Materials and Methods

### Participants

All participants provided informed consent for inclusion before participation in the study. Given the nature of the design of the study as a consumer research, no prior ethics approval was required. However, the study was conducted with the principles of the Declaration of Helsinki. Fifty-eight participants were consulted and selected for this study. Study participants were anonymized and assigned a unique participant id.

Participants completed an eligibility survey that included name, age, e-mail address, residential address. Participants confirmed that they were in good health and had no serious diagnoses or suspected medical condition. Inclusion criteria were for healthy participants older than 18 years old, willing to participate and able to provide informed consent. Exclusion criteria included not suffering from any significant health issues including any diagnosed mental health disorder, taking any medications including antibiotics or steroids treatment. Mental health disorders included clinically diagnosed anxiety, depression and/or stress.

### Intervention

Probiotic capsules containing HN001 at a dose of 9.5×10⁹ colony forming units (CFU) in an appropriate excipient were manufactured. Participants were given sufficient capsules for the 60-day study period. Accounting for the loss of viability of probiotics overtime participates received daily doses of between 6×10⁹ to 9.5×10⁹ CFU daily.

Before the start of the intervention, quality testing on the probiotic intervention to ensure gastric survivability and viability. An HN001 capsule was placed under stomach “like” conditions in a shaker flask. Bacteria were shaken for 15, 30, and 60 minutes at 37° C. and then tested for viability using standard flow cytometric methods. Briefly, probiotic samples were incubated with permeant (thiazole orange (TO)) and impermeant (propidium iodide (PI)) DNA binding dyes to stain for all cells and those with compromised membranes, respectively. Such that, when bacterial membrane is compromised, PI can enters the cell, whereas an intact viable cell can exclude it. Thus, a bacterial cell that observed to be double stained with both TO and PI can be considered injured when the membrane becomes completely disrupted allowing more PI into the cell, PI quenches the TO, such that dead bacterial cells become labelled only with PI. Using this method, it is possible to differentiate between live, injured, and dead cells based on the amount of each dye inside the cells.

### Outcomes

The primary outcome was the change in Oxford happiness questionnaire (OHQ) scores after 30 and 60 days. The Oxford Happiness Inventory (Argyle et al., 1989) was devised as a broad measure of personal happiness, mainly for in-house purposes at the Department of Experimental Psychology, University of Oxford in the late 1980s. However, since then, the questionnaire was widely used as reviewed by Argyle, Martin, and Lu in 1995 (Argyle et al., 1995). The scale has been found to behave consistently, and other workers have reported its use both in the UK (Furnham and Brewin, 1990; Joseph and Lewis, 1998) and the USA (Valiant, 1993). The OHQ has a total of 29 items. Each item is scored on a Likert scale from 1 to 6 (1=strongly disagree; 2=moderately disagree; 3=slightly disagree; 4=slightly agree; 5=moderately agree; 6=strongly agree). Some items are phrased positively and others negatively. Such that negative items (1, 3, 12, 13, 16, 18, 21 and 29) should be scored in reverse (Hills and Argyle, 2002). The total score is then is divided by 29. Interpretation of the final score is as follows: A final score between 1 to 2 is considered “not happy”; between 2 and 3 is “somewhat unhappy”; between 3 and 4 is considered “Neutral”; 4 is “somewhat or moderately happy”; between 4 and 5 is “rather happy”; between 5 and 6 is “very happy”; 6 is “too happy” (Hills and Argyle, 2002).

### Data Collection

The participants completed the OHQ prior to taking HN001 intervention (Timepoint 1), after taking HN001 for thirty (30) days (Timepoint 2), and after 60 days (Timepoint 3). Participants also answered questions regarding their age and gender.

### Statistical Analyses

Statistical analyses were performed using SPSS version 26 (Statistics, I., IBM Corp. Released 2013). All statistical tests were two sided at a 5% significance level. Repeated measures analyses of variance (time effect) was used to assess the effect of using the probiotics over time. Independent t test was used to compare the effect of gender on happiness scores.

### Results

### Initial Quality Testing and Gastric Survivability

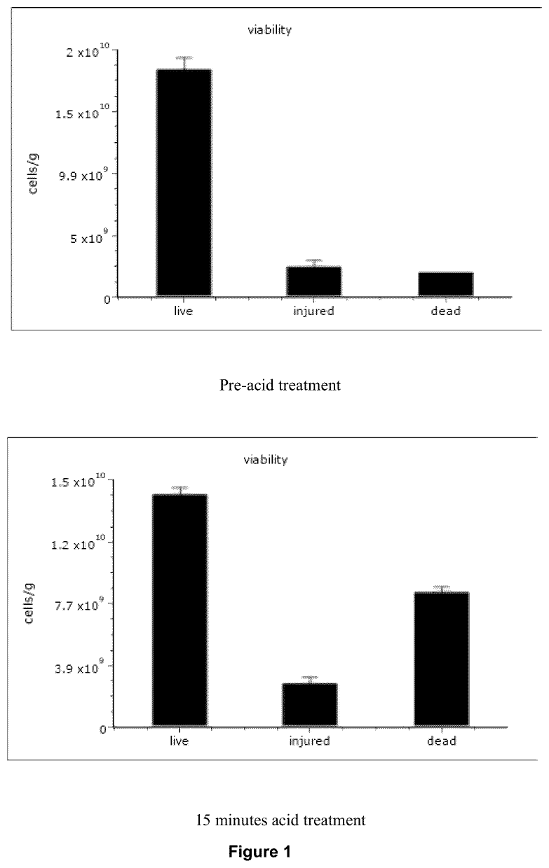

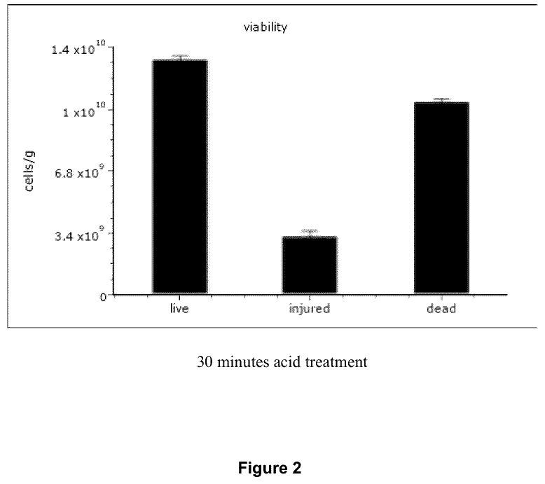

Flowcytometry testing, performed by BioForm Solutions, tested the live/injured/dead cell count over three intervals; pre-acid, after 15 minutes of the acid treatment; and 30 minutes after the acid treatment (FIGS. 1 and 2). Results indicated that HN001 remained viable following 30 minutes of exposure to acid at stomach pH. While levels of injured and dying cells increased over time, the drop in live cells was approximately 0.5 log 10, which is considered acceptable. Assuming that a probiotic capsule is dissolved quickly in stomach acid, and is taken with a small amount of liquid, then after 30 minutes, most (>67%) of that volume would have moved into the small intestine (Goyal et al., 2019).

### Recruitment

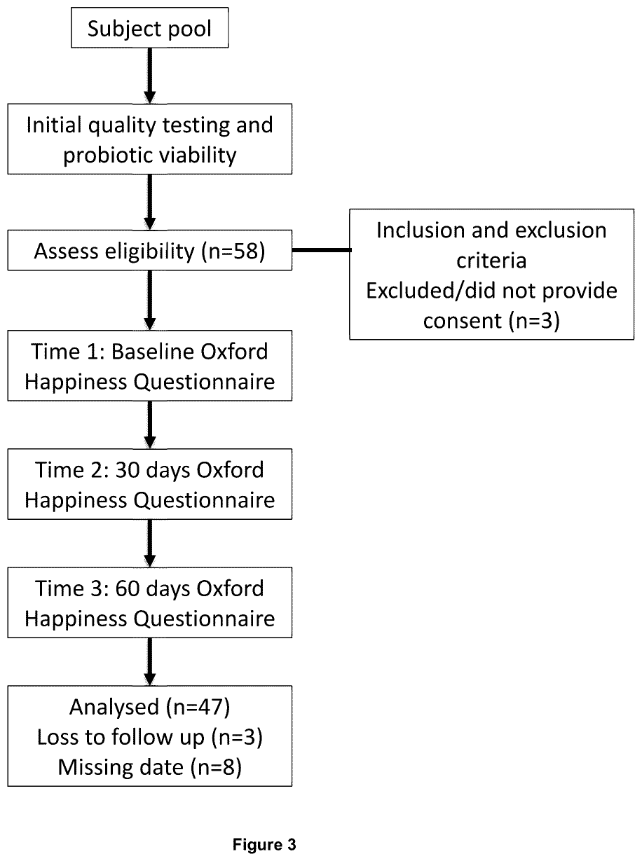

Volunteers (58) were selected to participate in this study. Each participant was required to complete an Eligibility Survey to confirm their ability to participate in this study and to ensure that the inclusion and exclusion criteria were met. FIG. 3 demonstrates the flowchart of the study. Three participants were a loss to follow up. Eight had missing data and were excluded from the final analyses. Therefore, the final analyses included 47 participants.

### Baseline Data

While a total of 58 participants were recruited to the study; 3 decided not to join the study and 8 participants had missing data and were subsequently removed from the study. Therefore 47 participants were analysed. Table 1 shows the demographic characteristics of the participants. Mean age of participants was 37.7 years ranging from 21 to 66 years. There were slightly more men than women in the study, but this difference was not statistically significant. Mean (sd) of happiness scores at base line was 4.29 (0.65) ranging from 2.97 to 5.55 (Table 2).

TABLE 1
Demographic characteristics of the participants (n = 47)

| No | Variable | Mean (sd¹) | N (%)² |
|---|---|---|---|
| 1 | Age (years) | 37.7 (10.12) | — |
| 2 | Gender | | |
| | a. Male | — | 24 (51.1) |
| | b. Female | — | 23 (48.9) |

### Outcomes

A summary of OHQ results at the 3 timepoints is shown in Table 2. Results showed clear increase in mean happiness scores over time and more people shifted above the neutral category over time. In fact, 72% of consumers improved their happiness scores after 30 days and 79% improved their happiness scores after 60 days when compared to the baseline. No participant was classified as unhappy at time points 2 or 3. The data was then categorized into unhappy and happy using “neutral and below” as unhappy and the “moderately happy” and “rather happy” as happy (Table 3). This categorization showed a similar trend for increased happiness.

TABLE 2
Mean happiness score and happiness groups at each time point (n = 47)

| No | Variable | Time 1 | Time 2 | Time 3 |
|---|---|---|---|---|
| 1 | Happiness scores | 4.29 (0.65) | 4.56 (0.61) | 4.61 (0.65) |
| | (Mean (sd¹)) | | | |
| 2 | Happiness groups | | | |
| | (n(%)²) | | | |
| | a. Not happy | 0 (0) | 0 (0) | 0 (0) |
| | b. Somewhat unhappy | 1 (2.1) | 0 (0) | 0 (0) |
| | c. Neutral | 13 (27.7) | 9 (19.1) | 8 (17) |
| | d. Moderately happy | 26 (55.3) | 27 (57.4) | 24 (51.1) |
| | e. Rather happy | 7 (14.9) | 11 (23.4) | 15 (31.9) |
| | f. Very happy | 0 (0) | 0 (0) | 0 (0) |
| | g. Too happy | 0 (0) | 0 (0) | 0 (0) |

¹sd = standard deviation;
²frequency (percentage)

TABLE 3
Prevalence of happiness at each time point (n = 47)

| No | Variable | Time 1 | Time 2 | Time 3 |
|---|---|---|---|---|
| 1 | Prevalence of Happiness | | | |
| | (n(%)²) | | | |
| | a. Happy | 33 (70.2) | 38 (80.9) | 39 (83) |
| | b. Unhappy | 14 (29.8) | 9 (19.1) | 8 (17) |

²frequency (percentage)

Independent t test showed that there was no significant difference between mean happiness scores between males and females (Table 4).

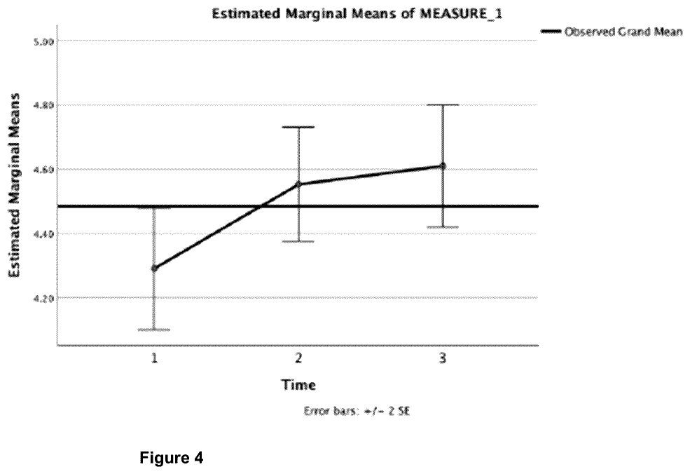

Using repeated measures ANOVA, results showed that there was a statistically significant increase in the happiness scores over time. Post hoc analyses using LSD showed that timepoints 2 and 3 were statistically different when compared with timepoint 1 whereas, no significant difference was observed between timepoints 2 and 3 (Table 5). FIG. 4 shows the mean plot and estimated marginal mean.

TABLE 4
Mean happiness comparison between Males
and females at each time point (n = 47)

| | | Mean difference | |
|---|---|---|---|
| No | Variable | (95% %)^{a} | P value^{b} |
| 1. | Mean Happiness Time 1 | 0.018 (0.19, −0.38) | 0.176 |
| 2. | Mean Happiness Time 2 | 0.19 (0.19, −0.19) | 0.325 |
| 3. | Mean Happiness Time 3 | 0.24 (0.18, −0.11) | 0.924 |

^{a}95% confidence interval;
ⁱndependent samples t test

TABLE 5
Repeated Measures ANOVA (n = 47)

| | | | | Grand | | | |
|---|---|---|---|---|---|---|---|
| | | | | Mean | | | |
| | | Wilk's | | (95% | | Effect | |
| No | Variable | Lamda | F^{a}(df^{b}) | C.I.)^{c} | Pvalue^{d} | size^{e} | Power |
| 1 | Happiness | 0.01 | 12.14 | 4.48 | <0.001^{f} | 0.21 | 0.99 |
| | scores | | (2) | (4.3, 4.7) | | | |

^{a}F statistics;
^{b}Degrees of freedom;
^{c}Grand mean (95% Confidence Interval);
^{d}Repeated Measures Analyses of variance;
^{e}Partial Eta Squared;
^{f}Pairwise comparison using LSD showed that Time 2 and 3 were statistically different when compared to Time 1 (mean happiness at time 2 and 3 were 4.55 and 4.61 respectively compared to mean happiness at time 1 which was 4.29)

### Conclusion

This study showed that HN001 survived the initial acidity test. This is an important factor in any good probiotic strain (Reid, 2005). Also, the study demonstrated that the participants had an increase in their happiness scores over time when compared to baseline.

### Example 2—Effect of Probiotics on Mental Well-Being and Happiness

The primary objective of the trial is to determine the effect of HN001 on the scores over a 4-week period using the Oxford happiness questionnaire (OHQ). The secondary objectives of the trial includes:
1. To determine the effect of HN001 on salivary cortisol level over 4 weeks period.
2. To determine the effect of HN001 on stress, anxiety and depression scores using DASS-21 over a 4 weeks period.

### Materials and Methods

### Trial Design

A 30 day double blinded placebo controlled randomized controlled trial.

### Sample and Sampling

Participants (120) will take park in the study (60 in the probiotics group: 60 in the placebo group). A simple randomization (1:1) will be used. The randomization plan will be kept by the Sponsor and will not be shared with the research team.

### Participants

This study aims to recruit 120 participants.

### Inclusion Criteria

Adults aged between 18-60 years old
Currently living in the US
Have access to a smart phone, internet and computer
Report moderate levels of perceived stress

Participants will be excluded if they
Are currently taking medication for depression or anxiety
Have had antibiotics within the previous month
Have an allergy to coconut

### Recruitment and Eligibility Screening

All eligibility screening, consent, and data collection will be managed via a secure web interface data collection platform facilitated by Survey Monkey. Participants will be recruited from a customer database. The advertisement will provide a link and QR code that interested people can use to access the Participant Information Sheet (PIS). Those who are interested in taking part will be asked to complete brief eligibility questions including a self-report stress questionnaire, the Perceived Stress Scale (“PSS”). The PSS is a 10-item questionnaire that asks people to rate how often in the preceding month they have felt stressed. Scores range from 0 to 40 with higher scores indicating higher stress levels. Eligible participants will be those with a score of 14 or higher.

Eligible participants will proceed to the electronic consent form. Following consent participants will answer demographic questions including contact details so they can be provided with the study products. The whole registration process will take roughly 10 minutes in one sitting.

### Baseline Data Collection

Once consent and demographic data collection is complete, participants will be sent a wearable Fitbit Charge 5 device, with instructions to wear it for two weeks to provide pre-study reference data. After the two-week Fitbit reference data collection period, participants will be sent a link to complete baseline questionnaires (Survey 1) and a posted home saliva cortisol test kit with instructions and postage paid return pack. At this time also, the gut microbiome testing will be done. Participants will be sent the investigational products at this time point.

### Intervention

Participants will be randomly assigned to receive HN001 probiotics capsules or placebo. Participants will be asked to take the capsule daily, with food or drink for 4 weeks. HN001 is the name for the probiotic strain. The placebo is coconut powder. Both the probiotics and placebo capsules are dairy and gluten free. Adherence Monitoring: Email or text messages (according to participant preference) will be sent at two, three, and four weeks to remind participants to take the supplement daily.

### Mid-Point Assessment

At the mid-point of the intervention (2 weeks after starting the supplement) participants will be sent out a link to the questionnaires (Survey 2).

### Post-Intervention Assessment

One week before the participant is due to complete the intervention, saliva collection kits will be sent out. Participants will be asked to collect the saliva sample and send it back using the pre-paid postage bag provided. Also, another microbiome testing will be done. At the end of the intervention period, participants will be sent an electronic link to complete the questionnaires (Survey 3).

### Questionnaires

The following questionnaires will be completed by study participants:

The Oxford Happiness Inventory (described above in Example 1) and the Depression Anxiety and Stress Scale DASS-21 (Lovibond and Lovibond, 1995).

### Salivary Cortisol

The saliva kit shall be included as part of the microbiome collection kit. The saliva kit contains a 15 ml sterile plastic tube with lid and instructions. The instructions outline that the participant will need to self-collect the saliva sample before going to bed by spitting into the tube to fill up ½ of the tube. The collection process should take no more than 15-30 minutes total. The participant shall write their name, the date, and the time of collection on the tube. The full sample volume will be used for measurement.

Fitbit Wearable Activity Data (from Wearable Device):

Participants will be asked to wear a Fitbit Charge 5 wristwatch for a two week baseline period and the duration of the 14 week intervention, 16 weeks in total. Participants will be asked to wear it at all times except for when washing or swimming for 14 weeks in total. Data extraction from the Fitbit will be cleaned and aggregated by a third party—DataMine. Raw data will be provided to the independent statistician through an encrypted USB stick for analysis.

### Microbiome

The microbiome kit contains 2 ml sterile plastic tubes with lid and instructions. The kit also includes gloves, a stool collector, and a pre-paid USPS mailer. The instructions outline that the participant will need to self-collect the stool sample. The collection process should take no more than 5 minutes total. The participant shall register the sample via a sample portal. The full sample volume will be used for measurement.

### Statistical Analyses

T-tests will analyse differences between intervention groups in psychological wellbeing, salivary cortisol, and objective physical activity from the Fitbit. A repeated measures analyses of covariate will also be used. Descriptive statistics will additionally be used.

### Results

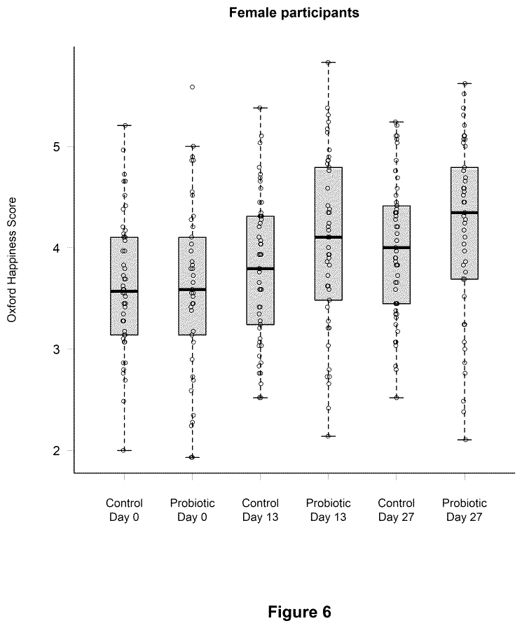

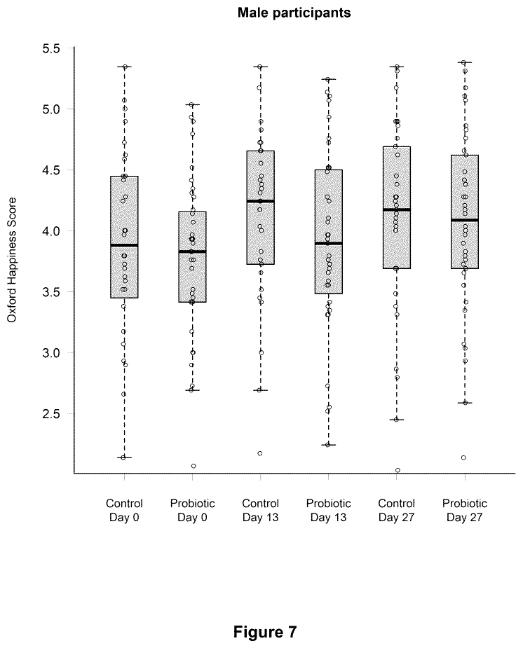

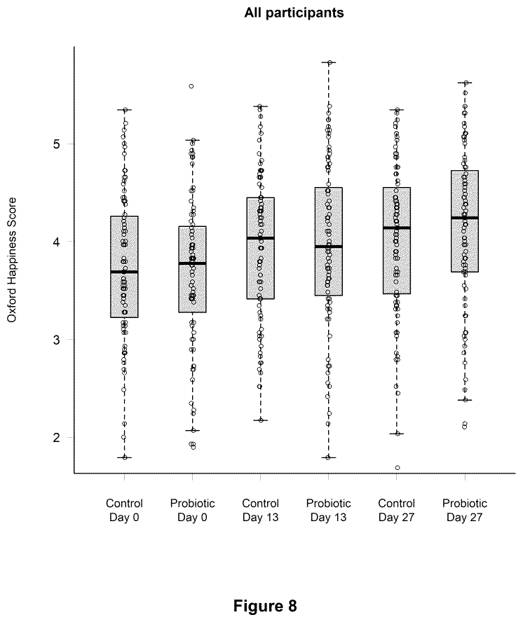

Results from the Oxford Happiness Inventory assessment are shown in FIG. 6, FIG. 7 and FIG. 8. In females the probiotic group had significantly higher happiness score, P=0.042 compared to the control group (FIG. 6). Many male subjects displayed improved happiness score, although the overall results were not statistically significant (P=0.88 was observed in the male cohort (FIG. 7)). It is noted that the male study included fewer participants than the female study. The assessment in males will be repeated in a larger male cohort. When the results from females and males are combined (FIG. 8). The probiotic group tended towards a higher happiness score, P=0.059.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

This application claims priority from Australian Provisional Application No. 2022903873 entitled “Methods for increasing mental well-being and/or happiness” filed on 16 Dec. 2022, the entire contents of which are hereby incorporated by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Argyle et al (1989) Recent advances in social psychology: An international perspective 189-203.
Argyle et al (1995) Stress and emotion 15:173-187.
Chatterjee et al (2020) Asian journal of psychiatry 51:102071.
Diener et al (1985) Journal of Personality Assessment 49:71-75.
FAO/WHO (2001) Evaluation of health and nutritional properties of powder milk and live lactic acid bacteria 1-4.
Fordyce (1988) Social indicators Research 20(4):355-381.
Furnham and Brewin (1990) Personality and individual differences 11:1093-1096.
GBD Mental Disorders Collaborators (2022) Global, regional, and national burden of 12 mental disorders in 204 countries and territories, 1990-2019: a systematic analysis for the Global Burden of Disease Study 2019. The Lancet Psychiatry.
Goyal et al (2019) Neurogastroenterology & Motility 31:e13546.
Hills and Argyle (2002) Personality and individual differences 33:1073-1082.
Joseph and Lewis (1998) Journal of clinical psychology 54:537-544.
Lovibond and Lovibond (1995) Manual for the Depression Anxiety & Stress Scales. (2nd Ed.) Sydney: Psychology Foundation.
Lyubomirsky and Lepper, (1999). Social Indicators Research 46:137-155.
Reid (2005) Current pharmaceutical design 11:11-16.
Rossouw et al (2021) PLoS One 16:e0259579.
Ryff (1989) Journal of Personality and Social Psychology, 57, 1069-1081.
Ryff et al (2007) National Survey of Midlife Development in the United States (MIDUS II), 2004-2006: Documentation of the Psychosocial Constructs and Composite Variables in MIDUS II Project 1. Ann Arbor, MI: Inter-university Consortium for Political and Social Research.
Sharma et al (2021) Current microbiology 78:449-463.
Sivamaruthi et al (2019) Tropical Journal of Pharmaceutical Research 18:889-895.
Slykerman et al (2017) EBioMedicine 24:159-165.
Statistics, I., IBM Corp. Released 2013. IBM SPSS Statistics for Windows, Version 26.0.
Stewart-Brown et al (2009) Health and quality of life outcomes, 7(1):15.
Tay et al (2020) Nutrients 12:3530.
Tennant et al (2007) Health and Quality of Life Outcomes, 5(1):63.
Valiant (1993) Personality and individual differences 15:447-453.
Vinderola et al (2022) Foods 11:1077.
Watson et al (1988) Journal of Personality and Social Psychology 54:1063-1070.
Zhou et al (2005) International Journal of Feed Microbiology 98:211-217.
Zółkiewicz et al (2020) Nutrients 12:2189.

1. A method of increasing happiness in a subject, the method comprising administering an effective amount of Lacticaseibacillus rhamnosus HN001 or a derivative or a component thereof to a subject.

2. The method of claim 1, wherein increasing happiness comprises treating unhappiness in a subject.

3. The method of claim 1, wherein happiness is assessed with one or more of the: Oxford Happiness Questionnaire (OHQ), Subjective Happiness Scale (SHS), Satisfaction with Life Scale (SLS), Panas Scale (PS), Authentic Happiness Inventory (AHI), Psychological Wellbeing Scale (PWS) and the Fordyce Emotions Questionnaire (FEQ).

4. The method of claim 1, wherein happiness is assessed with the Oxford Happiness Questionnaire (OHQ).

5. The method of claim 1, wherein administration comprises one or more of the following: i) daily administration, ii) administration for about 17 to about 80 days, iii) administration for about 30 or about 60 days, and iv) oral administration.

6-7. (canceled)

8. The method of claim 1, wherein the subject comprises one or more of the following features:
i) has not been treated for one or more of the following: anxiety, depression, stress, enhancing calm, occasional anxiety, postnatal depression and postnatal anxiety;
ii) is female;
iii) is male;
iv) is not pregnant, prenatal or postpartum;
v) has not been pregnant,
vi) is human,
vii) is a companion animal or a livestock animal, and
viii) is selected from an/a: infant, child, adolescent, adult and elderly adult.

9-12. (canceled)

13. The method of claim 1, wherein increasing happiness in a subject comprises changes in levels of salivary cortisol.

14. The method of claim 1, wherein increasing happiness in a subject does not comprise changes in levels of salivary cortisol.

15. The method of claim 1, wherein the method comprises administering about 6×10⁹ to about 1×10¹⁰ Lacticaseibacillus rhamnosus HN001 or a derivative thereof colony forming units (CFU) daily.

16. (canceled)

17. (canceled)

18. The method of claim 1, which further comprises administering one or more other probiotic or postbiotic microorganism/s.

19. The method of claim 18, wherein the one or more other probiotic or postbiotic microorganism/s are selected from one or more of:
i) selected from a: Acetilactobacillus, Agrilactobacillus, Akkermansia, Amylolactobacillus, Anaerobutyricum, Apilactobacillus, Bacillus, Bacteroides, Bombilactobacillus, Christensenella, Clostridium, Companilactobacillus, Dellaglioa, Enterococcus, Eubacterium, Faecalibacterium, Fructilactobacillus, Furfurilactobacillus, Holzapfelia, Lacticaseibacillus, Lactiplantibacillus, Lactococcus, Lapidilactobacillus, Latilactobacillus, Lentilactobacillus, Leuconostoc, Levilactobacillus, Ligilactobacillus, Limosilactobacillus, Liquorilactobacillus, Loigolactobacilus, Parabacteroides, Paucilactobacillus, Pediococcus, Prevotella, Schleiferilactobacillus, Secundilactobacillus, and Streptococcus;
ii) selected from a: Akkermansia muciniphila, Anaerobutyricum hallii, Bacillus coagulans, Bacillus subtilis, Bacteroides fragilis, Bacteroides thetaiotaomicron, Bacteroides uniformis, Bifidobacterium adolescentis, Bifidobacterium animalis, Bifidobacterium bifidum, Bifidobacterium breve, Bifidobacterium infantis, Bifidobacterium longum, Bifidobacterium longum, Christensenella minuta, Clostridium butyricum, Enterococcus durans, Enterococcus faecalis, Enterococcus faecium, Faecalibacterium prausnitzii, Lacticaseibacillus casei, Lacticaseibacillus paracasei, Lacticaseibacillus rhamnosus, Lactiplantibacillus pentosus, Lactiplantibacillus plantarum, Lactobacillus acidophilus, Lactobacillus bulgaricus, Lactobacillus crispatus, Lactobacillus gallinarum, Lactobacillus gasseri, Lactobacillus helveticus, Lactobacillus johnsonii, Lactococcus lactis, Latilactobacillus sakei, Lentilactobacillus kefiri, Leuconostoc mesenteroides, Levilactobacillus brevis, Ligilactobacillus salivarius, Limosilactobacillus fermentum, Limosilactobacillus reuteri, Parabacteroides goldsteinii, Pediococcus acidilactici, Pediococcus pentosaceus, Prevotella copri, Saccharomyces boulardii, and Streptococcus thermophilus;
iii) selected from: L. reuteri RC-14, B. longum BB536, L. lactis MG1363, L. plantarum LP299V, L. plantarum LP-115, B. lactis HN019, L. reuteri JCM 1112, L. casei ATCC393, B. animalis B94, L. paracasei LPC-37, L. acidophilus NCFM, B. lactis BL-04, B. lactis Bi-07, L. crispatus LBV88, L. gasseri LG-36, B. longum CCFM1029, L. rhamnosus HN067, L. rhamnosus GG (LGG), B. breve M-16V, B. lactis BB12 and L. acidophilus HN017; and
iv) a psychobiotic.

20-22. (canceled)

23. The method of claim 1, which further comprises administering one or more prebiotics optionally selected from one or more of: galacto-oligosaccharide, fructo-oligosaccharide, trans-galacto-oligosaccharide, inulin, a human milk oligosaccharide, gentio-oligosaccharide, xylo-oligosaccharide, isomalto-oligosaccharide, bran, resistant starch, lactulose, lactitol, flavanols and pectic oligosaccharide.

24-27. (canceled)

28. The method of claim 1, wherein Lacticaseibacillus rhamnosus HN001 or a derivative or a component thereof is in a form selected from a: tablet, powder, capsule, liquid, syrup, gummy and a sachet.

29. The method of claim 1, wherein Lacticaseibacillus rhamnosus HN001 or a derivative or a component thereof is administered in a nutritional supplement or food product.

30. The method of claim 29, wherein the food product is a dairy product.

31. The method of claim 30, wherein the dairy product is selected from: milk, yoghurt, drinking yoghurt, butter, cream, ice-cream and cheese.

32. The method of claim 29, wherein the food product or nutritional supplement is selected from: an infant formula, a follow-on formula, a growing-up formula, a maternal formula, a maternal supplement, an adult nutritional formulation, and an elderly nutritional formulation.

33. The method of claim 1, wherein Lacticaseibacillus rhamnosus HN001 is viable at the time of administration.

34-35. (canceled)

36. A method of increasing happiness in a subject, the method comprising administering an effective amount of Lacticaseibacillus rhamnosus comprising the AscI restriction enzyme digest profile of Lacticaseibacillus rhamnosus HN001 as shown in FIG. 5 or a component thereof to a subject.

37-38. (canceled)

39. A method of increasing mental well-being in a subject, the method comprising administering an effective amount of: Lacticaseibacillus rhamnosus HN001 or a derivative or a component thereof to a subject; or Lacticaseibacillus rhamnosus comprising the AscI restriction enzyme digest profile of Lacticaseibacillus rhamnosus HN001 as shown in FIG. 5 or a component thereof to a subject.

40-44. (canceled)
